(19) United States
(12) Patent Application Publication
(10) Pub. No.: US 2020/0185089 A1
(43) Pub. Date: Jun. 11, 2020

(54) Provider Matching Services

(71) Applicant: PreparedHealth, Inc., Chicago, IL (US)

(72) Inventors: Erin Karam, La Grange Park, IL (US); Ashish V. Shah, Glenview, IL (US); David M. Coyle, Chicago, IL (US)

(21) Appl. No.: 16/704,379

(22) Filed: Dec. 5, 2019

Related U.S. Application Data
(60) Provisional application No. 62/775,471, filed on Dec. 5, 2018.

(51) Int. Cl. G16H 40/20; G16H 10/60; G06F 16/29; G06Q 10/10

(52) U.S. Cl. CPC G16H 40/20; G06Q 10/1095; G06F 16/29; G16H 10/60

(57) ABSTRACT

Systems and methods for provider matching services in accordance with one or more embodiments are disclosed. In one embodiment, a method includes obtaining, from a request provider, patient data indicating a request for a provider for a patient, a geographic location of the patient's home, and at least one service requested for the patient, obtaining, from an availability engine, provider data indicating one or more providers in a geographic area, determining at least one relevant provider in the provider data based on the patient data, the geographic area, and the geographic location of the patient's home, determining an availability for each of the at least one relevant provider based on the at least one service requested for the patient, calculating a recommended provider for the patient based on the determined availability for each of the at least one relevant provider, and assigning the patient to the recommended provider.

### CROSS-REFERENCE TO RELATED APPLICATIONS

The instant application claims priority to U.S. Provisional Patent Application No. 62/775,471, filed Dec. 5, 2018 and titled “Provider Matching Services,” the disclosure of which is hereby incorporated by reference in its entirety.

### FIELD

The present disclosure generally relates to data processing and more specifically the dynamic allocation of resources based on service availability.

### BACKGROUND

Many patients require services on release from the hospital. A social worker at the hospital auctions off a patient to a facility by sending requests to different facilities with the first facility to accept a request is assigned the patient. This results in patients being assigned to a facility without regard to quality, patient-specific needs, ranking, availability, or responsiveness. Accordingly, there is a need for a system that can assign patients to a facility with regard to quality, patient-specific needs, ranking, availability, and/or responsiveness.

### SUMMARY

The following presents a simplified summary of various embodiments described herein. This summary is not an extensive overview, and is not intended to identify key or critical elements or to delineate the scope of the claims. The following summary merely presents some concepts in a simplified form as an introductory prelude to the more detailed description provided below. Corresponding apparatus, systems, and computer-readable media are also within the scope of the disclosure.

Systems and methods for provider matching services in accordance with one or more embodiments are disclosed. In one embodiment, a method includes obtaining, by a provider matching server system and from a request provider, patient data indicating a request for a provider for a patient, a geographic location of the patient's home, and at least one service requested for the patient, obtaining, by the provider matching server system and from an availability engine, provider data indicating one or more providers in a geographic area, determining, by the provider matching server system, at least one relevant provider in the provider data based on the patient data, the geographic area, and the geographic location of the patient's home, determining, by the provider matching server system, an availability for each of the at least one relevant provider based on the at least one service requested for the patient, calculating, by the provider matching server system, a recommended provider for the patient based on the determined availability for each of the at least one relevant provider, and assigning, by the provider matching server system, the patient to the recommended provider.

In yet other embodiments, the method further includes automatically scheduling, by the provider matching server system, at least one appointment with the provider for the patient, wherein each of the at least one appointment is for each of the at least one service requested for the patient.

In still other embodiments, the method further includes determining, by the provider matching server system, the at least one relevant provider based on a distance between the provider and the geographic location of the patient's home.

In yet still other embodiments, the method further includes determining, by the provider matching server system, the availability of a provider to provide the at least one service requested for the patient based on a predicted future availability of the provider to provide the at least one service requested for the patient.

In yet other additional embodiments, the method further includes determining, by the provider matching server system, the at least one relevant provider based on an insurance provider indicated in the patient data.

In still other additional embodiments, the method further includes determining, by the provider matching server system, the at least one relevant provider based on a patient-specific score for each provider in the one or more providers.

In yet still other additional embodiments, the method further includes calculating, by the provider matching server system, the patient specific score for a specific provider by determining, by the provider matching server system, resource availability at a particular time for the specific provider, wherein the resource corresponds to at least one service requested for the patient, determining, by the provider matching server system, historical performance data for the specific provider, and calculating, by the provider matching server system, the patient-specific score for the specific provider based on the resource availability and the historical performance data.

Yet other embodiments include a provider matching device including a processor and a memory in communication with the processor and storing instructions that, when executed by the processor, cause the provider matching device to obtain, from a request provider, patient data indicating a request for a provider for a patient, a geographic location of the patient's home, and at least one service requested for the patient, obtain, from an availability engine, provider data indicating one or more providers in a geographic area, determine at least one relevant provider in the provider data based on the patient data, the geographic area, and the geographic location of the patient's home, determine an availability for each of the at least one relevant provider based on the at least one service requested for the patient, calculate a recommended provider for the patient based on the determined availability for each of the at least one relevant provider, and assign the patient to the recommended provider.

In yet other embodiments, the instructions, when executed by the processor, further cause the provider matching device to automatically schedule at least one appointment with the provider for the patient, wherein each of the at least one appointment is for each of the at least one service requested for the patient.

In still other embodiments, the instructions, when executed by the processor, further cause the provider matching device to determine the at least one relevant provider based on a distance between the provider and the geographic location of the patient's home.

In yet still other embodiments, the instructions, when executed by the processor, further cause the provider matching device to determine the availability of a provider to provide the at least one service requested for the patient based on a predicted future availability of the provider to provide the at least one service requested for the patient.

In yet other additional embodiments, the instructions, when executed by the processor, further cause the provider matching device to determine the at least one relevant provider based on an insurance provider indicated in the patient data.

In still other additional embodiments, the instructions, when executed by the processor, further cause the provider matching device to determine the at least one relevant provider based on a patient-specific score for each provider in the one or more providers.

In yet still other additional embodiment, the instructions, when executed by the processor, further cause the provider matching device to calculate the patient specific score for a specific provider by determine resource availability at a particular time for the specific provider, wherein the resource corresponds to the at least one service indicated in the patient data, determine historical performance data for the specific provider, and calculate the patient-specific score for the specific provider based on the resource availability and the historical performance data.

Still other embodiments include a non-transitory machine-readable medium storing instructions that, when executed by one or more processors, cause the one or more processors to perform steps including obtaining, from a request provider, patient data indicating a request for a provider for a patient, a geographic location of the patient's home, and at least one service requested for the patient, obtaining, from an availability engine, provider data indicating one or more providers in a geographic area, determining at least one relevant provider in the provider data based on the patient data, the geographic area, and the geographic location of the patient's home, determining an availability for each of the at least one relevant provider based on the at least one service requested for the patient, calculating a recommended provider for the patient based on the determined availability for each of the at least one relevant provider, and assigning the patient to the recommended provider.

In yet other embodiments, the instructions, when executed by one or more processors, further cause the one or more processors to perform steps including automatically scheduling at least one appointment with the provider for the patient, wherein each of the at least one appointment is for each of the at least one service requested for the patient.

In still other embodiments, the instructions, when executed by one or more processors, further cause the one or more processors to perform steps including determining the at least one relevant provider based on a distance between the provider and the geographic location of the patient's home.

In yet still other embodiments, the instructions, when executed by one or more processors, further cause the one or more processors to perform steps including determining the availability of a provider to provide the at least one service requested for the patient based on a predicted future availability of the provider to provide the at least one service requested for the patient.

In yet other additional embodiments, the instructions, when executed by one or more processors, further cause the one or more processors to perform steps including determining the at least one relevant provider based on an insurance provider indicated in the patient data.

In still other additional embodiments, the instructions, when executed by one or more processors, further cause the one or more processors to perform steps including determining the at least one relevant provider based on a patient-specific score for each provider in the one or more providers, wherein calculating the patient specific score for a specific provider includes determining resource availability at a particular time for the specific provider, wherein the resource corresponds to the at least one service requested for the patient, determining historical performance data for the specific provider, and calculating the patient-specific score for the specific provider based on the resource availability and the historical performance data.

Other objects, advantages and novel features, and further scope of applicability of the present disclosure will be set forth in part in the detailed description to follow, and in part will become apparent to those skilled in the art upon examination of the following, or can be learned by practice of the disclosed embodiments. The objects and advantages of the present disclosure can be realized and attained by means of the instrumentalities and combinations particularly pointed out in the claims.

### BRIEF DESCRIPTION OF THE DRAWINGS

The description will be more fully understood with reference to the following figures, which are presented as exemplary embodiments of the disclosure and should not be construed as a complete recitation of the scope of the disclosure, wherein:

FIG. 1 is a conceptual illustration of a provider matching system in accordance with an embodiment of the disclosure;

FIG. 2A is a conceptual illustration of a provider matching device in accordance with an embodiment of the disclosure;

FIG. 2B is a conceptual illustration of a provider matching server system in accordance with an embodiment of the disclosure;

FIG. 3 is a conceptual illustration of a data flow within a provider matching system in accordance with an embodiment of the disclosure;

FIG. 4A is a flow chart conceptually illustrating a process for matching patients to providers in accordance with an embodiment of the disclosure;

FIG. 4B is a flow chart conceptually illustrating a process for rating providers in accordance with an embodiment of the disclosure; and

FIGS. 5A-C are user interface screenshots in accordance with embodiments of the disclosure.

### DETAILED DESCRIPTION

Turning now to the drawings, systems and methods for provider matching systems in accordance with various embodiments are disclosed. Provider matching systems allow for the recommendation of patients to service providers (such as outpatient medical facilities) based on real-time predictions of available capacity at one or more providers. Capacity can be determined over an arbitrary time period, such as the next twelve hours, twenty-four hours, two days, one week, one month, etc. By dynamically identifying available capacity, patients can be assigned to facilities that are able to see the patient, provide appropriate services for the patient, are covered by the patient's insurance, and/or service a particular geographic region. The geographic region can be filtered based on a particular radius, neighborhood characteristics, insurance restrictions, and/or any other criteria. In a variety of embodiments, the geographic region of the service area includes street-level detail of the patient and/or the provider location to determine which providers should be included in the recommendation. Providers can be rated using a combination of filters and ranks. In a variety of embodiments, providers can be ranked according to scores calculated on a per-patient basis. Provider scores can be determined based on availability, services available (e.g. languages spoken, facilities, access to equipment, affiliations with healthcare providers, etc.), specialties provided, performance objectives (e.g. turnaround times for particular services), patient preferences, and/or a number of other factors. In many embodiments, patient data and/or provider data can be provided by third-party server systems, such as healthcare service registration system and/or an electronic medical record (EMR) server system. In this way, provider matching systems allow for improved provider matching that results in patients being assigned to one or more facilities based on provider rankings, availability, and responsiveness by processing data to improve the functionality of a computer system itself. The facilities can include any of a variety of post-acute care facilities, skilled nursing facilities, home health services, personal care services, and/or community-based services.

A variety of provider matching systems and provider matching processes in accordance with a variety of embodiments are described in more detail herein.

### Provider Matching Systems

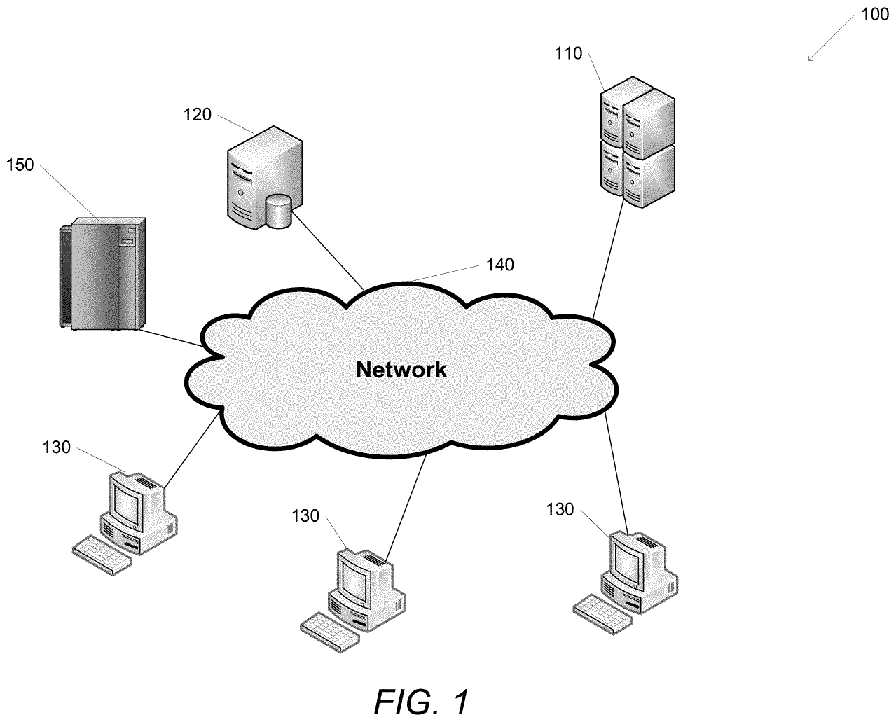

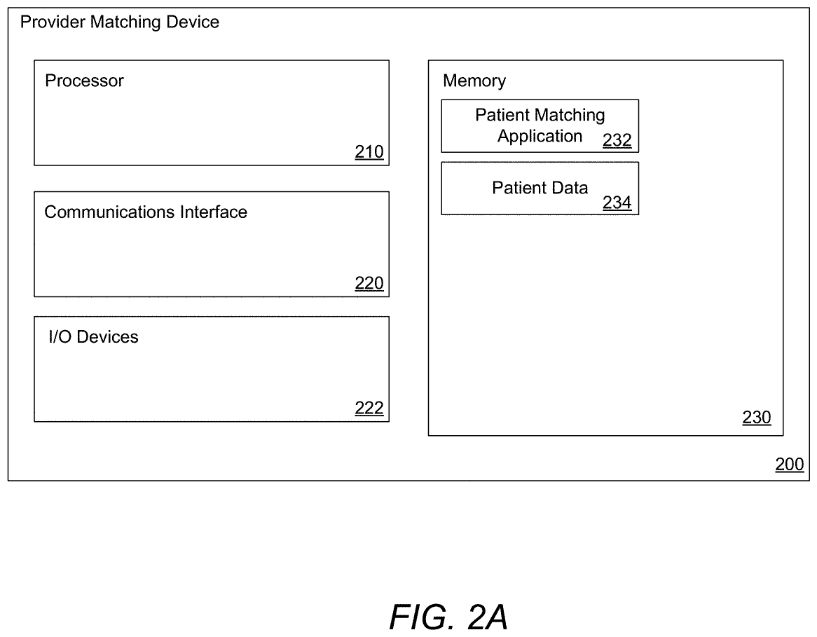

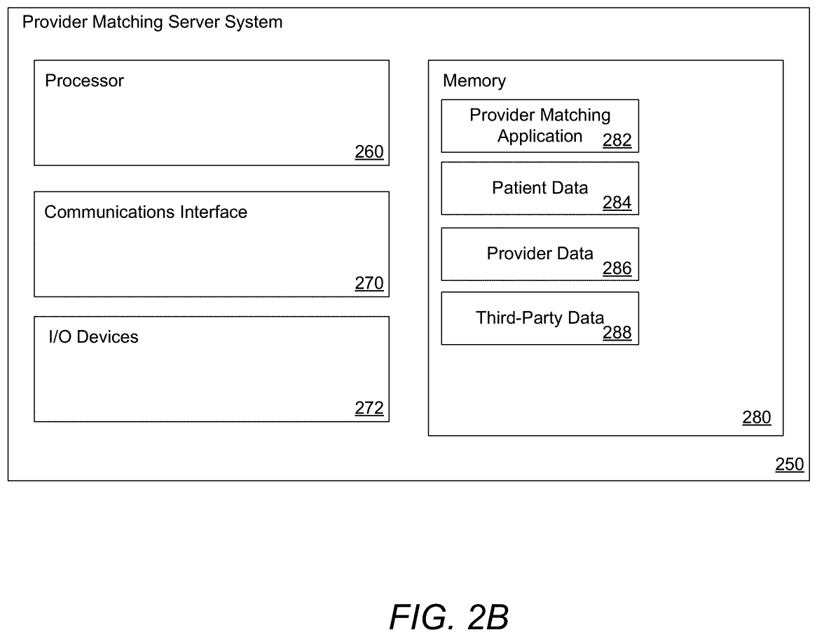

Provider matching systems in accordance with embodiments can assign patients to provider facilities based on the attributes of the provider facility, the patient's needs, and the availability of the provider facility. A conceptual illustration of a provider matching system in accordance with an embodiment is shown in FIG. 1A. The provider matching system 100 includes provider matching server system 110, provider server system 120, and provider matching devices 130 connected via network 140. In several embodiments, the provider matching system 100 includes a third-party server system 150 connected to the network 140. In many embodiments, the provider matching server system 110 and/or provider server system 120 are implemented using a single server. In a variety of embodiments, provider matching server system 110 and/or provider server system 120 are implemented using a plurality of servers. In many embodiments, provider matching devices 130 are implemented utilizing provider matching server system 110 and/or provider server system 120. Network 140 can be one or more of a variety of networks, including, but not limited to, wide-area networks, local area networks, and/or the Internet as appropriate to the requirements of specific applications in accordance with various embodiments. Any of the devices and systems described herein can be implemented, in whole or in part, using one or more computing devices described with respect to FIGS. 2A-B.

Provider matching server system 110 can obtain and store a variety of data from any of a variety of sources and any of a variety of providers of data, such as third-party server systems 150, as appropriate to the requirements of specific applications. Provider matching server system 110 can obtain requests to match patients to providers from the provider matching devices 130. The provider matching system 110 can determine the availability of relevant providers for a particular request and match a patient request to an available provider.

The data transferred to and from various computing devices in provider matching system 100 can include secure and sensitive data, such as confidential documents, customer personally identifiable information, and account data. Therefore, it can be desirable to protect transmissions of such data using secure network protocols and encryption, and/or to protect the integrity of the data when stored on the various computing devices. A file-based integration scheme or a service-based integration scheme can be utilized for transmitting data between the various computing devices. Data can be transmitted using various network communication protocols. Secure data transmission protocols and/or encryption can be used in file transfers to protect the integrity of the data such as, but not limited to, File Transfer Protocol (FTP), Secure File Transfer Protocol (SFTP), and/or Pretty Good Privacy (PGP) encryption. In many embodiments, one or more web services can be implemented within the various computing devices. Web services can be accessed by authorized external devices and users to support input, extraction, and manipulation of data between the various computing devices in the provider matching system 100. Web services built to support a personalized display system can be cross-domain and/or cross-platform, and can be built for enterprise use. Data can be transmitted using the Secure Sockets Layer (SSL) or Transport Layer Security (TLS) protocol to provide secure connections between the computing devices. Web services can be implemented using the WS-Security standard, providing for secure SOAP messages using XML encryption. Specialized hardware can be used to provide secure web services. Secure network appliances can include built-in features such as hardware-accelerated SSL and HTTPS, WS-Security, and/or firewalls. Such specialized hardware can be installed and configured in the provider matching system 100 in front of one or more computing devices such that any external devices can communicate directly with the specialized hardware.

Provider matching systems in accordance with one or more embodiments are described above with respect to FIG. 1; however, it should be appreciated that any of a number of variations can be utilized in accordance with one or more embodiments. In several embodiments, provider matching server systems, third-party server systems, provider server systems, and/or provider matching devices provide an interface, such as an application programming interface (API) or web service, to transmit and receive some or all of the data for further processing. Access to the interface can be open and/or secured using any of a variety of techniques, such as by using client authorization keys, as appropriate to the requirements of specific applications.

### Provider Matching Devices and Server Systems

Provider matching systems in accordance with one or more embodiments include a variety of devices for obtaining patient data, provider data, and matching patients to providers. A conceptual illustration of a provider matching device in accordance with an embodiment is shown in FIG. 2A. The provider matching device 200 includes a processor 210 in communication with memory 230. The provider matching device 200 can also include one or more communication interfaces 220 capable of sending and receiving data and input/output (I/O) devices 222 capable of obtaining a variety of input data. In a number of embodiments, the communication interface 220 and/or I/O devices 222 are in communication with the processor 210 and/or the memory 230. In several embodiments, the memory 230 is any form of storage storing a variety of data, including, but not limited to, a patient matching application 232 and patient data 234. In many embodiments, patient matching application 232 and/or patient data 234 are stored using an external server system and received by the provider matching device 200 using the communications interface 220.

A conceptual illustration of a provider matching server system in accordance with an embodiment is shown in FIG. 2B. The provider matching server system 250 includes a processor 260 in communication with memory 280. The provider matching server system 250 can also include one or more communications interfaces 270 capable of sending and receiving data with a variety of devices, such as with third-party server systems and provider matching devices. Provider matching server system 250 can also include input/output (I/O) devices 272 capable of obtaining a variety of input data. In a number of embodiments, the communication interface 270 and/or I/O devices 272 are in communication with the processor 260 and/or the memory 280. In several embodiments, the memory 280 is any form of storage storing a variety of data, including, but not limited to, a provider matching application 282, patient data 284, provider data 286, and/or third party data 288. In many embodiments, the provider matching application 282, patient data 284, provider data 286, and/or third party data 288 are stored using an external server system and received by the task tracking server system 250 using the communications interface 270.

Input/output (I/O) devices can include a microphone, keypad, touch screen, and/or stylus through which a user of a computing device can provide input, and can also include one or more of a speaker for providing audio output and a video display device for providing textual, audiovisual, and/or graphical output. Memory can store software used by a computing device, such as an operating system, application programs, and/or databases. The various hardware memory units in the illustrated memories can include volatile and nonvolatile, removable and non-removable media implemented in any method or technology for storage of information such as computer-readable instructions, data structures, program modules, or other data. Memory can include one or more physical persistent memory devices and/or one or more non-persistent memory devices including, but not limited to, random access memory (RAM), read only memory (ROM), electronically erasable programmable read only memory (EEPROM), flash memory or other memory technology, optical disk storage, magnetic cassettes, magnetic tape, magnetic disk storage or other magnetic storage devices, or any other medium that can be used to store the desired information and that can be accessed by a processor or any other device. Communication interfaces can include one or more transceivers, digital signal processors, and/or additional circuitry and software for communicating via any network, wired or wireless, using any protocol as described herein. It will be appreciated that the network connections shown are illustrative and any means of establishing a communications link between the computers can be used using any of various network protocols such as TCP/IP, Ethernet, FTP, HTTP and the like, and be performed using any of a variety of wireless communication technologies such as GSM, CDMA, WiFi, and LTE.

The processor 210 and processor 260 can be directed, by the patient matching application 232 and the provider matching application 282 respectively, to perform a variety of provider matching processes described herein. Some or all of the data described herein can be stored using one or more databases. Databases can include, but are not limited to relational databases, hierarchical databases, distributed databases, in-memory databases, flat file databases, XML databases, NoSQL databases, graph databases, and/or any combination thereof. Various elements within memory or other components can include one or more caches including, but not limited to, CPU caches used by a processor, page caches used by an operating system, disk caches of a hard drive, and/or database caches used to cache content from a database. For embodiments including a CPU cache, the CPU cache can be used by one or more processors to reduce memory latency and access time. A processor can retrieve data from or write data to the CPU cache rather than reading/writing to memory, which can improve the speed of these operations. In some examples, a database cache can be created in which certain data from a database is cached in a separate smaller database in a memory separate from the database, such as in RAM or on a separate computing device. For instance, in a multi-tiered application, a database cache on an application server can reduce data retrieval and data manipulation time by not needing to communicate over a network with a back-end database server. These types of caches and others can be included in various embodiments, and can provide potential advantages in certain implementations of devices, systems, and methods described herein, such as faster response times and less dependence on network conditions when transmitting and receiving data.

Although specific architectures for provider matching devices and provider matching server systems in accordance with one or more embodiments are conceptually illustrated in FIGS. 2A-B, any of a variety of architectures, including those that store data or applications on disk or some other form of storage and are loaded into memory at runtime, can also be utilized. Additionally, any of the data utilized in the system can be cached and transmitted once a network connection (such as a wireless network connection via the communications interface) becomes available. In a variety of embodiments, a memory includes circuitry such as, but not limited to, memory cells constructed using transistors, that store instructions. Similarly, a processor can include logic gates formed from transistors (or any other device) that dynamically perform actions based on the instructions stored in the memory. In several embodiments, the instructions are embodied in a configuration of logic gates within the processor to implement and/or perform actions described by the instructions. In this way, the systems and methods described herein can be performed utilizing both general-purpose computing hardware and by single-purpose devices.

### Provider Matching Processes

Provider matching processes can include obtaining data from a number of data feeds and processing that data using one or more data feeds in order to provide facility recommendations to a patient. In a number of embodiments, provider server systems and/or third-party server systems can provide data feeds describing the patients and/or providers. In a variety of embodiments, a third-party server system can provide electronic medical records for a patient. In many embodiments, a third-party server system can provide admission, discharge, and/or transfer information for patients assigned to a provider. Third-party data can be provided continuously and/or on demand and/or can be updated in real-time. In this way, (near) real-time performance and/or available capacity for a provider can be determined based on the needs of a particular patient.

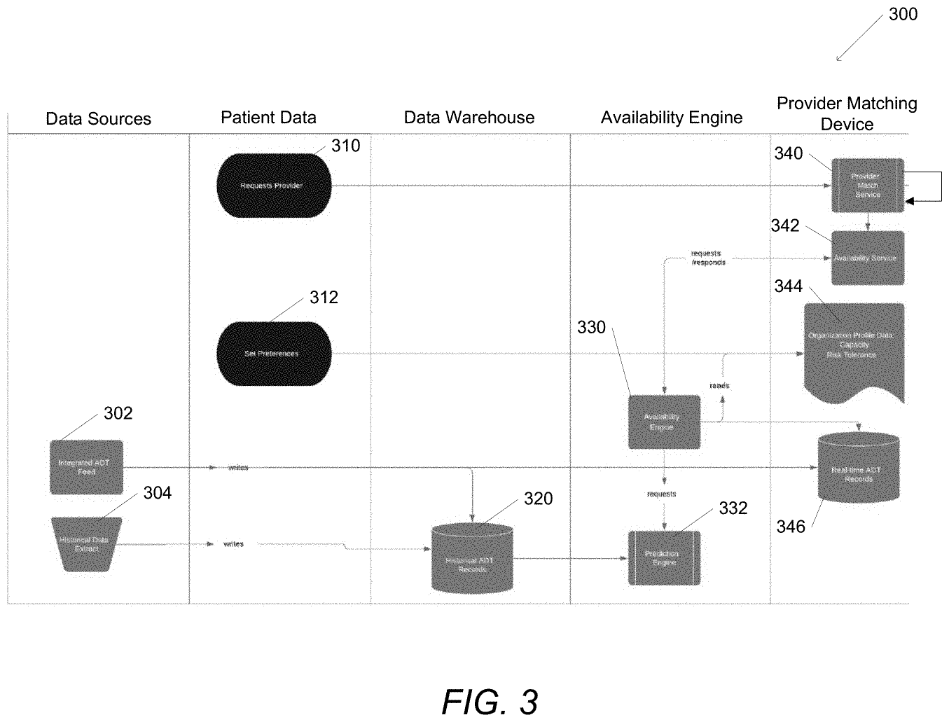

FIG. 3 is a conceptual illustration of a data flow 300 within a provider matching system in accordance with an embodiment. The data flow 300 can include a variety of data sources, such as an integrated admit-discharge-transfer (ADT) feed 302 and/or a historical data extract 304. The integrated ADT feed 302 can provide data regarding when patients is admitted to a hospital, transferred to another facility, and/or discharged from the hospital, along with health and treatment information regarding the patients. Historical data extract 304 can include a variety of data regarding previous admittances, facility assignments, and/or any other patient data as described herein. The data flow 300 can also include a variety of patient data, such as request provider 310 and/or patient preferences 312. The data flow 300 can also include a data warehouse storing historical ADT records 320. The data warehouse can include any databases and/or combination of databases as described herein. The historical ADT records can include, for example, ADT information regarding one or more patients obtained from the integrated ADT feed 302 and/or historical data extract 304. The data flow 300 can also include an availability engine 330 and/or prediction engine 332. The prediction engine 332 can use historical ADT records to determine predicted availability at one or more providers. The availability engine 330 can request availability predictions from prediction engine 332 and provide a variety of availability information to provider matching devices. As shown in data flow 300, provider matching devices can include a provider matching service 340 that processes provider requests provided by a request provider 310 and provides the requests to availability service 342. Availability service 342 can obtain availability information for one or more providers indicated in an availability requests from availability engine 330. The provider matching device can also include a variety of facility data 344, such as facility profile data, capacity information, and risk information. In several embodiments, the provider matching device includes a real-time ADT record database 346 storing information from the integrated ADT feed 302. In many embodiments, the availability engine 330 can obtain ADT information from the real-time ADT record database 346 and/or provide facility availability data to the real-time ADT record database 346 for storage.

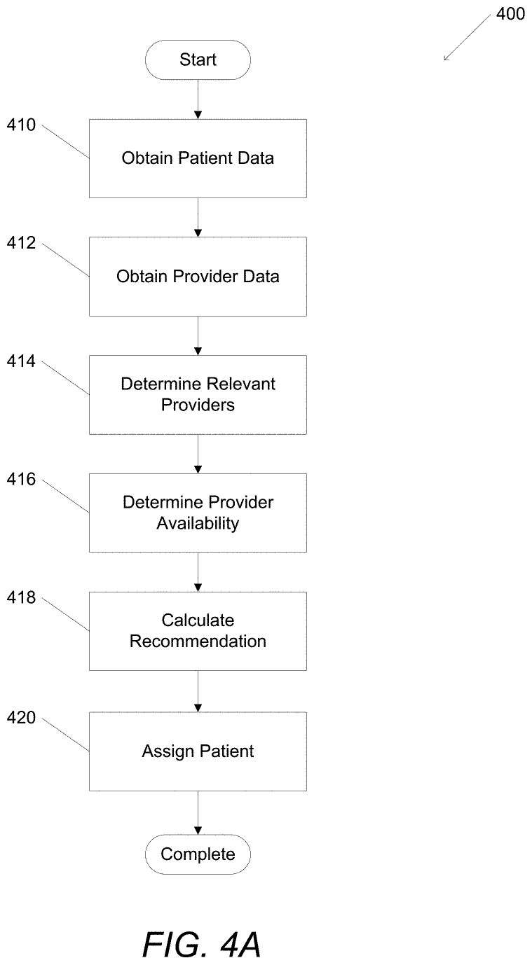

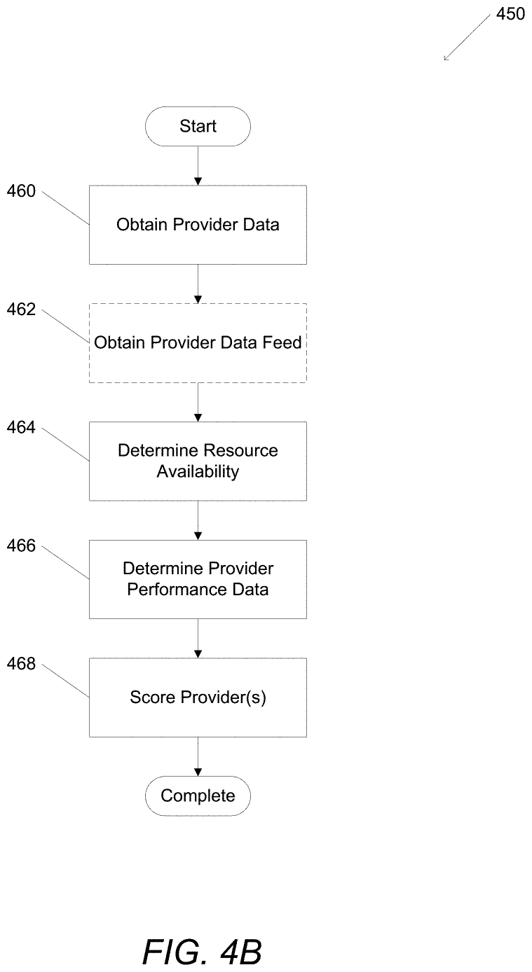

Provider matching processes can include matching providers and patients based on the patient's needs and the attributes of the provider. FIG. 4A is a flow chart conceptually illustrating a process for matching patients to providers in accordance with an embodiment. The process 400 can include obtaining (410) patient data, obtaining (412) provider data, and determining (414) relevant providers. Patient data can be based on medical and/or non-medical needs, such as the patient's home address, the location of the provider facility, the service area of the provider facility, languages spoken by the patient and/or service provider, the patient's insurance coverage, personal preferences, home environment, and any of a variety of other factors. In several embodiments, patient needs can be determined based on a recommendation engine. In a variety of embodiments, the recommendation engine recommends additional treatments and/or services based on the patient data. Geographic locations can be based on an absolute location (such as a location obtained using a GPS) and/or an address. Provider data can include a variety of attributes describing a provider. Provider attributes can include services provided by the provider, equipment provided by the provider, the provider's capacity (e.g. number of beds available), patient throughput, patients recently assigned to the provider, insurance policies accepted by the provider, performance objectives of the provider, average length of stay, peer rakings, exact service area of the provider down to the street level, and/or a variety of other factors. Relevant providers can include those providers having attributes matching one or more of the medical and/or non-medical needs of a patient. For example, a relevant provider can include any provider that provides at least one service needed by the patient that is within a particular geographic range of the patient's home and accepts the patient's insurance. In several embodiments, the relevancy of particular providers can be determined based on a provider score as described in more detail with respect to FIG. 4B.

Provider availability can be determined (416), a recommendation can be calculated (418), and a patient can be assigned (420). In a variety of embodiments, provider availability can be determined by querying an availability engine that provides (near) real-time and/or projected availability for one or more of the relevant providers. For example, provider availability can be indicated for one week after a patient is discharged from a hospital. A recommendation can be calculated based on any of a variety of factors, including provider availability, provider scores, distances from the patient's home to the provider, etc. One or more providers can be indicated in the recommendation. The recommendation can be responsive to a request for a provider for a particular patient, such as those made by patients themselves and/or hospital employees on behalf of the patient as part of the patient's discharge from the hospital. A patient can be assigned to a provider based on the calculated recommendation. Once assigned, a patient can have one or more appointments automatically scheduled with the provider such that the patient can receive requested and/or necessary services from the provider.

A variety of provider matching processes can include determining relevant providers by scoring one or more providers. FIG. 4B is a flow chart conceptually illustrating a process for rating providers in accordance with an embodiment. The process 450 includes obtaining (460) provider data and, in a variety of embodiments, obtaining (462) a provider data feed. Provider data feeds can be obtained from third-party server systems and/or from provider server systems as appropriate to the requirements of specific applications of one or more embodiments. In several embodiments, a provider data feed can provide historical and/or real-time availability information for a provider.

Resource availability can be determined (464) and/or provider performance data can be determined (466). Resource availability at a provider can be predicted and/or provided in real-time and can be based on the availability of resources at (and/or over) a particular period and/or third party data inputs. The capacity of a provider can be based on the resources (e.g. equipment, beds, etc.) available at the provider and/or the number of patients assigned to the provider based on their expected usage of those resources. For example, a provider can be a five-star provider for patients with congestive heart failure and have an average length of stay of less than five days. In many embodiments, a provider is scored based on a proximity score calculated based on the patient's address, the location of the provider's facility, and/or a service area.

Providers can be scored (468). In a variety of embodiments, at least one provider can be scored for a particular patient. The scores can be calculated based on the patient data, additional data provided by the patient and/or third-party server systems, and/or the provider attributes. Scores can be on a per-provider and/or a per-patient basis. The scored providers can also be filtered based on the patient's needs such that providers that do not provide adequate services and/or performance are not scored. The providers can be ranked based on their scores for the particular patient and an appropriate provider can be assigned to the patient.

A variety of data flows and provider matching processes are shown and described with respect to FIG. 3 and FIGS. 4A-B; however, it should be noted that any of a variety of data and processes, including those that use more or less data than described herein and/or those that utilize alternative rating mechanisms for providers, can be utilized as appropriate to the specific requirements of various embodiments.

### User Interfaces

A variety of user interfaces can be utilized to obtain data and/or match patients to providers using a variety of provider matching processes. A referral can be generated for a patient based on the patient's data. A set of matching providers can be presented. In a number of embodiments, the providers are presented based on a provider score generated based on the patient data.

In many embodiments, a map can be presented showing the location of the patient and/or provider(s). The location can also include the patient's clinical and/or non-clinical needs, which can be generated using a recommendation engine. An indication of preferred providers can be displayed. Providers can be preferred based on the provider's performance, insurance policies, the patient's preferences, and/or a variety of other factors. Insurance policies accepted by the provider can be indicated in the results. Different providers can be presented on the map using different indicators based on the attributes of the provider. Providers can be ranked and/or filtered based on their rating (either overall or with respect to one or more services provided by the provider), the provider's location and/or service area, and any other search criteria as appropriate. In many embodiments, the service area of one or more service providers is indicated on the map.

In a number of embodiments, a provider search can be provided. In several embodiments, a search score is generated for one or more providers based on the patient's needs, provider attributes, search terms, and/or a variety of other factors. The search score for a provider can be weighted based on particular attributes and/or search terms. For example, a provider's search score can be increased by two if the provider matches the patient's insurance policy, while the score can be increased by one of the provider is a trusted partner and within a twenty mile radius of the patient's location. The search results can be dynamically updated based on a geographic area displayed in the user interface.

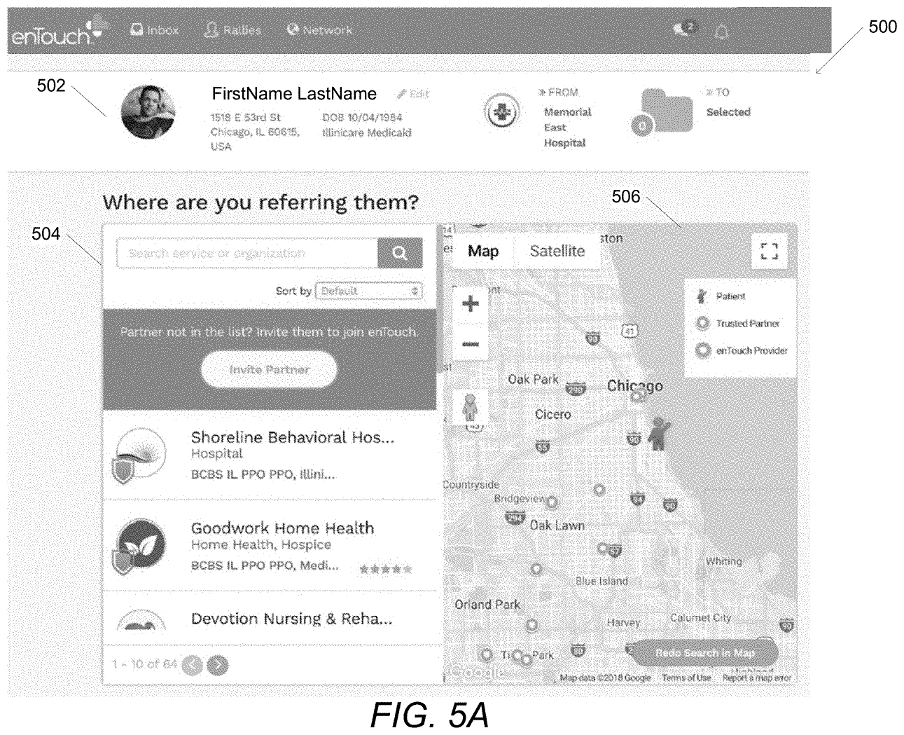

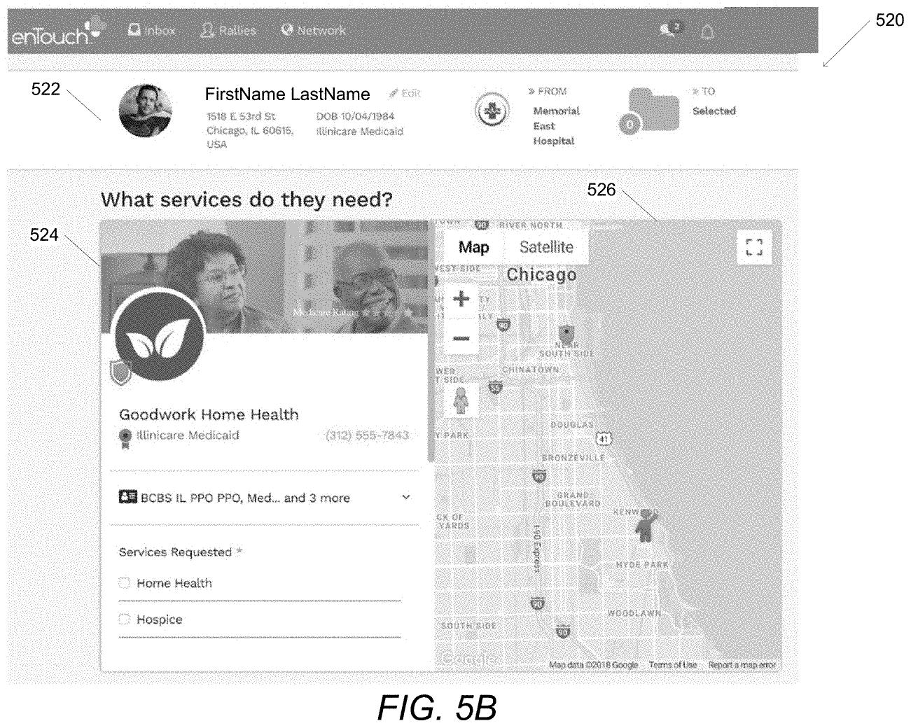

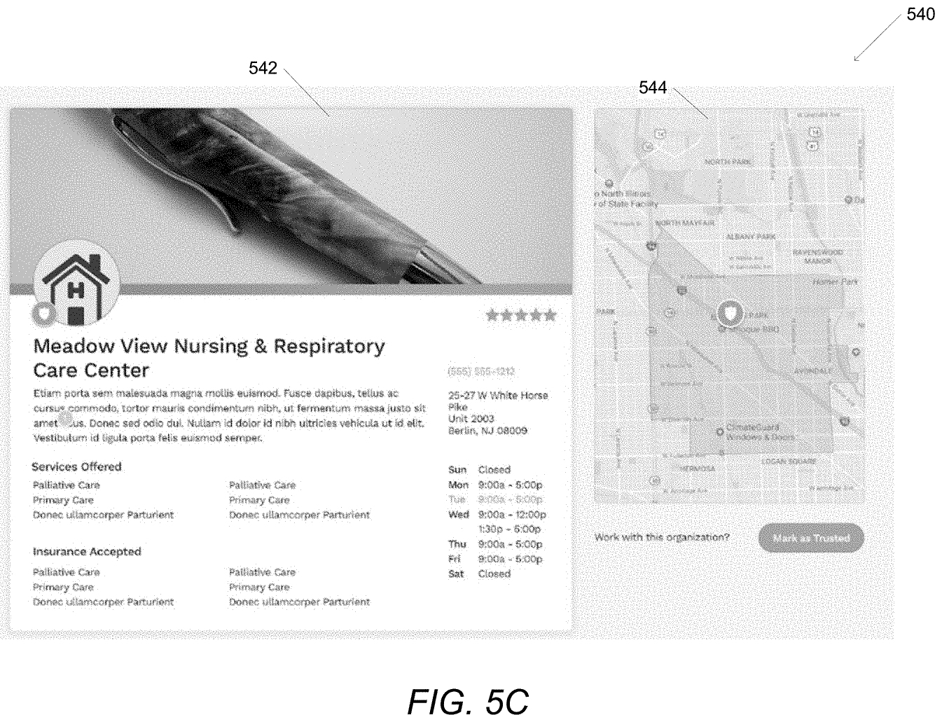

FIGS. 5A-C are user interface screenshots for a provider matching system in accordance with one or more embodiments. Turning now to FIG. 5A, a user interface 500 for locating relevant service providers is shown. The user interface 500 includes patient data 502 indicating that the referral is being generated for patient FirstName LastName who is being discharged from Memorial East Hospital. A variety of other patient data, including the patient's home address, date of birth, and insurance provider is shown. However, it should be noted that more or less patient data can be included in user interface 500 as appropriate. User interface 500 further includes a listing of relevant providers 504 for the indicated patient and a map view 506 that visually illustrates the patient's home location and markers indicating the location of each of the relevant providers. The listing of relevant providers 504 includes a prompt for inviting a particular provider that does not appear in the listing of relevant providers 504. The icons used to represent each provider in the map view 506 can be selected based on one or more attributes associated with the provider. For example, trusted providers can be indicated with a special icon, while the most recommended provider can be indicated with a star icon.

Turning now to FIG. 5B, a user interface 520 for viewing provider information is shown. The user interface 520 includes patient data 522 as described herein. The user interface 520 further includes provider details 524. The provider details 524 can include any data regarding a selected provider, such as the name of the provider, an indication of if the provider accepts the patient's insurance, the provider's contact information, other insurances accepted by the provider, and/or a listing of requested services provided by the provider. User interface 520 can also include a map view 526 visually illustrating the patient's home location and the location of the selected provider.

Turning now to FIG. 5C, a user interface 540 for showing provider data is shown. The user interface 540 includes provider data 542 and a map view 544. The provider data 542 can include a variety of detailed provider data for a provider including, but not limited to, the provider's name, a full listing of services provided by the provider, a full listing of insurances accepted by the provider, the provider's contact information, the provider's address, and the provider's hours of operation. The map view 544 can include a map centered on the provider's address and showing the provider's service area. The provider's service area can be indicated by highlighting a geographic area on the map. The geographic area can be any arbitrary geographic area as indicated by the provider.

A variety of user interfaces are shown and described with respect to FIGS. 5A-C; however, it should be noted that any of a variety of user interfaces can be utilized as appropriate to the specific requirements of various embodiments.

One or more embodiments discussed herein can be embodied in computer-usable or readable data and/or computer-executable instructions, such as in one or more program modules, executed by one or more computers or other devices as described herein. Generally, program modules include routines, programs, objects, components, data structures, and the like that perform particular tasks or implement particular abstract data types when executed by a processor in a computer or other device. The modules can be written in a source code programming language that is subsequently compiled for execution, or can be written in a scripting language such as (but not limited to) HTML or XML. The computer executable instructions can be stored on a computer readable medium such as a hard disk, optical disk, removable storage media, solid-state memory, RAM, and the like. As will be appreciated by one of skill in the art, the functionality of the program modules can be combined or distributed as desired in various embodiments. In addition, the functionality can be embodied, in whole or in part, in firmware or hardware equivalents such as integrated circuits, field programmable gate arrays (FPGA), and the like. Particular data structures can be used to implement one or more embodiments discussed herein, and such data structures are contemplated within the scope of computer executable instructions and computer-usable data described herein. Various embodiments discussed herein can be embodied as a method, a computing device, a system, and/or a computer program product.

Although the present disclosure has been described in certain specific aspects, many additional modifications and variations would be apparent to those skilled in the art. In particular, any of the various processes described above can be performed in alternative sequences and/or in parallel (on the same or on different computing devices) in order to achieve similar results in a manner that is more appropriate to the requirements of a specific application. It is therefore to be understood that the present disclosure can be practiced otherwise than specifically described without departing from the scope and spirit of the present disclosure. Thus, embodiments of the present disclosure should be considered in all respects as illustrative and not restrictive. It will be evident to the annotator skilled in the art to freely combine several or all of the embodiments discussed here as deemed suitable for a specific application of the disclosure. Throughout this disclosure, terms like “advantageous”, “exemplary” or “preferred” indicate elements or dimensions which are particularly suitable (but not essential) to the disclosure or an embodiment thereof, and can be modified wherever deemed suitable by the skilled annotator, except where expressly required. Accordingly, the scope of the disclosure should be determined not by the embodiments illustrated, but by the appended claims and their equivalents.

What is claimed is:

1. A method, comprising:
obtaining, by a provider matching server system and from a request provider, patient data indicating a request for a provider for a patient, a geographic location of the patient's home, and at least one service requested for the patient;
obtaining, by the provider matching server system and from an availability engine, provider data indicating one or more providers in a geographic area;
determining, by the provider matching server system, at least one relevant provider in the provider data based on the patient data, the geographic area, and the geographic location of the patient's home;
determining, by the provider matching server system, an availability for each of the at least one relevant provider based on the at least one service requested for the patient;
calculating, by the provider matching server system, a recommended provider for the patient based on the determined availability for each of the at least one relevant provider; and
assigning, by the provider matching server system, the patient to the recommended provider.

2. The method of claim 1, further comprising automatically scheduling, by the provider matching server system, at least one appointment with the provider for the patient, wherein each of the at least one appointment is for each of the at least one service requested for the patient.

3. The method of claim 1, further comprising determining, by the provider matching server system, the at least one relevant provider based on a distance between the provider and the geographic location of the patient's home.

4. The method of claim 1, further comprising determining, by the provider matching server system, the availability of a provider to provide the at least one service requested for the patient based on a predicted future availability of the provider to provide the at least one service requested for the patient.

5. The method of claim 1, further comprising determining, by the provider matching server system, the at least one relevant provider based on an insurance provider indicated in the patient data.

6. The method of claim 1, further comprising determining, by the provider matching server system, the at least one relevant provider based on a patient-specific score for each provider in the one or more providers.

7. The method of claim 6, further comprising calculating, by the provider matching server system, the patient specific score for a specific provider by:
determining, by the provider matching server system, resource availability at a particular time for the specific provider, wherein the resource corresponds to at least one service requested for the patient;
determining, by the provider matching server system, historical performance data for the specific provider; and
calculating, by the provider matching server system, the patient-specific score for the specific provider based on the resource availability and the historical performance data.

8. A provider matching device, comprising:
a processor; and
a memory in communication with the processor and storing instructions that, when executed by the processor, cause the provider matching device to:
obtain, from a request provider, patient data indicating a request for a provider for a patient, a geographic location of the patient's home, and at least one service requested for the patient;
obtain, from an availability engine, provider data indicating one or more providers in a geographic area;
determine at least one relevant provider in the provider data based on the patient data, the geographic area, and the geographic location of the patient's home;
determine an availability for each of the at least one relevant provider based on the at least one service requested for the patient;
calculate a recommended provider for the patient based on the determined availability for each of the at least one relevant provider; and
assign the patient to the recommended provider.

9. The provider matching device of claim 8, wherein the instructions, when executed by the processor, further cause the provider matching device to automatically schedule at least one appointment with the provider for the patient, wherein each of the at least one appointment is for each of the at least one service requested for the patient.

10. The provider matching device of claim 8, wherein the instructions, when executed by the processor, further cause the provider matching device to determine the at least one relevant provider based on a distance between the provider and the geographic location of the patient's home.

11. The provider matching device of claim 8, wherein the instructions, when executed by the processor, further cause the provider matching device to determine the availability of a provider to provide the at least one service requested for the patient based on a predicted future availability of the provider to provide the at least one service requested for the patient.

12. The provider matching device of claim 8, wherein the instructions, when executed by the processor, further cause the provider matching device to determine the at least one relevant provider based on an insurance provider indicated in the patient data.

13. The provider matching device of claim 8, wherein the instructions, when executed by the processor, further cause the provider matching device to determine the at least one relevant provider based on a patient-specific score for each provider in the one or more providers.

14. The provider matching device of claim 13, wherein the instructions, when executed by the processor, further cause the provider matching device to calculate the patient specific score for a specific provider by:
determine resource availability at a particular time for the specific provider, wherein the resource corresponds to the at least one service indicated in the patient data;
determine historical performance data for the specific provider; and
calculate the patient-specific score for the specific provider based on the resource availability and the historical performance data.

15. A non-transitory machine-readable medium storing instructions that, when executed by one or more processors, cause the one or more processors to perform steps comprising:
obtaining, from a request provider, patient data indicating a request for a provider for a patient, a geographic location of the patient's home, and at least one service requested for the patient;
obtaining, from an availability engine, provider data indicating one or more providers in a geographic area;
determining at least one relevant provider in the provider data based on the patient data, the geographic area, and the geographic location of the patient's home;
determining an availability for each of the at least one relevant provider based on the at least one service requested for the patient;
calculating a recommended provider for the patient based on the determined availability for each of the at least one relevant provider; and
assigning the patient to the recommended provider.

16. The non-transitory machine-readable medium of claim 15, wherein the instructions, when executed by one or more processors, further cause the one or more processors to perform steps comprising automatically scheduling at least one appointment with the provider for the patient, wherein each of the at least one appointment is for each of the at least one service requested for the patient.

17. The non-transitory machine-readable medium of claim 15, wherein the instructions, when executed by one or more processors, further cause the one or more processors to perform steps comprising determining the at least one relevant provider based on a distance between the provider and the geographic location of the patient's home.

18. The non-transitory machine-readable medium of claim 15, wherein the instructions, when executed by one or more processors, further cause the one or more processors to perform steps comprising determining the availability of a provider to provide the at least one service requested for the patient based on a predicted future availability of the provider to provide the at least one service requested for the patient.

19. The non-transitory machine-readable medium of claim 15, wherein the instructions, when executed by one or more processors, further cause the one or more processors to perform steps comprising determining the at least one relevant provider based on an insurance provider indicated in the patient data.

20. The non-transitory machine-readable medium of claim 15, wherein the instructions, when executed by one or more processors, further cause the one or more processors to perform steps comprising determining the at least one relevant provider based on a patient-specific score for each provider in the one or more providers, wherein calculating the patient specific score for a specific provider comprises:
determining resource availability at a particular time for the specific provider, wherein the resource corresponds to the at least one service requested for the patient;
determining historical performance data for the specific provider; and
calculating the patient-specific score for the specific provider based on the resource availability and the historical performance data.
